(19) United States
(12) Patent Application Publication
(10) Pub. No.: US 2023/0414836 A1
(43) Pub. Date: Dec. 28, 2023

(54) DEVICES TO BE IMPLANTED INTO THE HUMAN BODY

(71) Applicant: TECRES S.p.A, Sommacampagna (Verona) (IT)

(72) Inventors: Giovanni FACCIOLI, Monzambano (MN) (IT); Renzo SOFFIATTI, Nogara (VR) (IT)

(21) Appl. No.: 18/243,439

(22) Filed: Sep. 7, 2023

Related U.S. Application Data
(63) Continuation-in-part of application No. 15/551,497, filed on Aug. 16, 2017.

(30) Foreign Application Priority Data
Feb. 16, 2015 (IT) VR2015A000022

(51) Int. Cl. A61L 27/44; A61L 27/54; A61L 27/56

(52) U.S. Cl. CPC A61L 27/446; A61L 27/54; A61L 27/56

(57) ABSTRACT

Temporary and/or disposable device that can be implanted in the human body, including a biocompatible material implantable in the human body and including a plastic material, wherein the material includes a pharmaceutical or medical substance including at least one antibiotic and a radio-opacifying agent; method for the obtainment of such device and thread of 3D-printing material for the obtainment of such device.

### TECHNICAL FIELD OF THE INVENTION

The present invention concerns devices able to be implanted in the human body such as catheters, screws or joints, couplings or meshes, spacer devices, prostheses or prosthetic devices, etc. made of a biocompatible and implantable material. Furthermore, the present invention concerns a method for moulding such material to produce such devices with a 3D printing machine.

### STATE OF THE ART

The technology of additive production or three-dimensional printing of objects is currently becoming increasingly popular.

In particular, three-dimensional or 3D printing, which represents a natural evolution of 2D printing, allows an object to be reproduced from a three-dimensional model through the superimposition of successive layers of material, until the desired shape is obtained. Such a three-dimensional model is obtained through suitable software.

3D printers are generally faster, more reliable and simpler to use with respect to other technologies for additive production and, moreover, offer the possibility of moulding and assembling parts made up of different materials with different physical and mechanical properties in a single construction process.

The operation of a three-dimensional printer, according to an exemplificative version, is based on the use of a 3D file developed by suitable software.

The 3D model of the object of interest is prepared—through the aforementioned software—in a series of portions or layers in cross section thereof.

Each portion or layer is then printed one onto the other, in order to recreate the entire three-dimensional object.

There are different 3D printing technologies; their main differences concern, among others, the way in which the various layers are printed.

Some methods, in order to produce the various layers, use materials that melt or soften. Some examples of such technology are “Selective Laser Sintering” (SLS) or “Direct Metal Laser Sintering” (DMLS) or “Fused Deposition Modeling” (FDM).

Other methods provide for the deposition layer-by-layer of liquid materials, which are made to set with different technologies. An example of such a technology is known as “Digital Light Processing” (DLP).

Furthermore, in the case of lamination systems there are thin layers that are cut according to the desired shape and then joined together through known techniques.

A first 3D printing method consists of a printing system by extrusion of material. The printer creates the model one layer at a time, spreading a layer of powder (plaster or resins) and distributing a binder thereupon, for example printing like with an inkjet.

The process is repeated layer by layer until the desired shape of the object is obtained. In this way, it is possible to make projections in the finished product.

FDM technology, used in conventional quick prototyping, provides for a nozzle adapted for depositing a molten polymer layer-by-layer on a support structure.

Furthermore, some 3D printers for additive synthesis use a thread of thermoplastic polymer as construction material.

In the field of orthopaedics, three-dimensional printing technology has led to bone or parts thereof being made. For example, it has been possible to implant a cranium printed in 3D to a patient.

The skullcap was made with a special resin through the use of a 3D printer. Other recently-developed prostheses were obtained through three-dimensional printing of a titanium-based material.

The materials usually used for 3D printing are: plastic materials, for example thermoplastic polymers (for example for SLS and PDM), metals, sand, glass (for example for SLS), photopolymers (for example for stereolithography), laminated sheets (often of the paper type) and relative glues, titanium alloys (for example for “Electron beam melting” or EBM), resins, clays, ceramic, etc.

In particular, for jet or thread 3D printing, the material used must be melted and extrudable through a nozzle suitable for the purpose.

Therefore, one of the problems to be solved in order to be able to three-dimensionally print some objects, particularly for medical or orthopaedic use, is precisely that of making materials having the appropriate characteristics for the final purpose for which the object must be made and at the same time characteristics suitable for the selected three-dimensional printing method.

Basically, surgical practice provides for implanting resins or plastic materials in the body for a short time (for example for catheters) or permanently (for example for polymethyl methacrylate or PMMA, constituent of bone cement, for ultra high molecular weight polyethylene or UHMWPE or in short PE, as friction surface in hip, knee, shoulder prostheses etc., or for polyetheretherketone or PEEK, as cranial prostheses, spinal cages, etc.).

Except for the PMMA of bone cement, all of the other plastic materials undergo mechanical processing in order to obtain a prosthesis or a device to be implanted in the human body.

This occurs both due to objective difficulty in moulding those specific plastic materials, and particularly for the extremely heterogeneous demand of prostheses or devices to be implanted in the human body of greatly different shape and size, requirements which only mechanical processing can quickly satisfy.

However, these plastic materials (for example PE and PEEK) do not and cannot contain pharmaceutical or medical substances, like for example one or more antibiotics, nor radio-opacifying agents, like for example barium, nor possible further medical additives used to treat the patient or for surgical treatment.

Such substances, however, would be very useful and appreciated inside such plastics in order to carry out a healing function (for example local antibiotic) and be easily detected radiographically, as done on the other hand by antibiotic-loaded bone cement provided with radio-opacifying agents.

Therefore, there is a need for a biocompatible and implantable material, added to with pharmaceutical or medical substances and/or radio-opacifying agents and/or other useful additives, to be used for moulding, also three-dimensional printing moulding, of devices to be implanted in the human body or spacer devices.

The application WO2012/007535 discloses a part for endosseous implantation molded through injection moulding of a material comprising a thermoplastic binder (preferably PEEK) that incorporate fibers; TCP and zeolite can be incorporated into the binder, the latter substance being able to confer radiopacity at the implant.

The application EP0472237 discloses a material comprising UHMWPE and inorganic filler such as calcium phosphate or hydroxyapatite.

The application DE102007052519 discloses a medical implant, comprising a polymeric material (such as polyethylene or polypropylene) and an antimicrobial composition (including silicon dioxide and metal-containing nanoparticles).

The application WO2010/096053 discloses a medical implant incorporating a medical substance, such as silver or penicillin.

The application WO2013/184010 discloses a middle ear prosthesis including silver powder.

The U.S. Pat. No. 6,641,831 discloses a non-degradable medical product comprising at least two substances: substance A and substance B, wherein substance A is more lipophilic than substance B and has a given solubility in water, lower than that of substance B. At least one, among substance A and substance B, is a pharmaceutically active agent, i.e. an antimicrobial substance.

### SUMMARY OF THE INVENTION

The task of the present invention is to improve the state of the art.

In such a technical task, an aim of the present invention is to provide a biocompatible and implantable material, adapted for being used in moulding technology, possibly also three-dimensional printing, and which can be added to with pharmaceutical or medical substances and/or with further additives. An aim of the present invention is to provide a biocompatible and implantable material that is extrudable.

In accordance with an aspect of the present invention, a temporary and/or disposable device to be implanted in the human body or a spacer device for the treatment of a bone or joint location made with a biocompatible and implantable material is provided, according to the present application.

In accordance with another aspect of the present invention, a biocompatible and implantable material is provided according to the present application.

An advantage of such a device to be implanted in the human body or a spacer device for the treatment of a bone or joint seat consists of being able to be added to with pharmaceutical or medical substances or with additives and be made through moulding, possibly three-dimensional moulding.

A further advantage of the device to be implanted in the human body or a spacer device for the treatment of a bone or joint location consists of being able to be personalised or made in series in a quick and simple manner, substantially without the need for further surface finishing processing.

In accordance with another aspect of the present invention a method for obtaining a biocompatible and implantable material is provided, adapted for being used in printing technology, possibly also three-dimensional printing, and which can be added to with pharmaceutical or medical substances and/or with a radio-opacifying agent and/or with further additives, according to the present application.

An advantage of such a method is that it is quick and simple, substantially without the need for further surface finishing processing steps.

The present application refers to preferred and advantageous embodiments of the invention.

### DETAILED DESCRIPTION OF THE INVENTION

The present invention refers to a device to be implanted in the human body or a spacer device for the treatment of a bone or joint location made with a biocompatible material implantable in the human body. Specifically, the device of the present invention is temporary and/or disposable.

Such a material can comprise one or more of the following materials: an acrylic resin or a plastic material or polyethylene (PE) or low density polyethylene or high density polyethylene or ultra high molecular weight polyethylene (UHMWPE) or polypropylene or polyamide or polyetheretherketone (PEEK), or a mixture thereof or a plastic material selected to have a melting point lower than the degradation temperature of at least one antibiotic.

Such plastic materials, in a version of the invention, are insoluble.

In a further version of the invention, the plastic material is soluble and comprises, for example, polylactic or polyglycolic acid polymers or other suitable polymers.

Possible acrylic resins include an acrylic copolymer made up of MMA, styrene and ethyl-acrylate or polymethyl methacrylate or mixtures comprising acrylic polymers and/or copolymers.

One of the main characteristics of such a material is that it is mouldable, for example through injection moulding or through three-dimensional printing or through forming presses or through thermoplastic moulding technology.

In this way, especially when it concerns three-dimensional printing, it is possible to mould such a material according to a simple and quick procedure, capable of providing both medical devices or spacer devices produced in series, and personalised devices. For example, the molding step may comprise feeding at least one thread of material to a tridimensional printer in order to obtain the device according to the present invention.

In the latter case, indeed, it is possible to obtain a three-dimensional model of the device to be obtained, by selecting the size and shape or configuration most suitable for the surgical or anatomical requirements of the patient, and produce the relative device adapted to the specific patient.

In the aforementioned cases, however, thanks to the mouldability of the material according to the present invention, it is possible to obtain finished devices, substantially without the need to carry out further surface finishing or processing steps thereof.

The material according to the present invention also comprises at least one additive such as a pharmaceutical or medical substance and a radio-opacifying agent and/or a further additive. The pharmaceutical or medical substance comprised in the material according to the present invention can comprise or consist of at least one antibiotic, for example gentamicin sulphate or another suitable antibiotic, such as one of more of the following: Amikacin, Azlocillin, Aztreonam, Clarithromycin, Chloramphenicol, Ciprofloxacin, Clindamycin, Coumermycin, Fosfomycin, Josamycin, Kanamycin, Mezlocillin, Mupirocin, Nalidixic acid, Netilmicin, Norfloxacin, Novobiocin, Ofloxacin, Oxacillin, Sulbactam, Tobramycin, Trimethoprim, Trimethoprim+Sulphamethoxazole, Vancomycin, another suitable antibiotic which is thermoresistant and that is active against microbial infections, etc.

The pharmaceutical or medical substance comprised in the material according to the present invention can further comprise or consist of at least an antiseptic agent, of organic or inorganic nature, a bacteriostatic agent, like for example silver in its various forms, such as metallic powder or salts comprising citrate, proteinate, colloidal, electrolytic, or other forms that can be used in the human body, boric acid, etcetera.

For example, if the antiseptic agent or the bacteriostatic agent is metallic silver, this is insensitive to the melting temperature of the plastic material that would receive it according to the present invention.

Moreover, variously salified silver, for example colloidal silver powder, is insensitive to the melting temperature only of a few plastic materials of the material according to the present invention.

Possible bacteriostatic agents also comprise copper and/or gold, as well as the aforementioned silver, in their various forms, for example their salts or components. Such materials are, indeed, thermostable. Other thermostable inorganic substances having a medicating action can be advantageously included in the molten plastic material. A further example is boric acid that has an antiseptic action and is thermostable at over 300° C.

Such an option is particularly relevant when the device to be obtained with said material is a spacer device for treating an infection present in a bone or joint location.

The function of spacer devices, indeed, is to maintain the joint space left by an infected prosthesis, which for this reason is removed, and at the same time to treat the infection of the bone location, internally comprising for example a pharmaceutical or medical substance, like for example at least one antibiotic, to be eluted in the area to be treated. The material according to the present invention, in addition, could also comprise a radio-opacifying agent, like for example metallic powders or heavy metals powers, for example of tungsten, tantalum, silver or a metal salt of barium sulphate, zirconium oxide, bismuth oxide, etcetera.

The radio-opacifying agent can comprise, in addition or as an alternative option, at least one iodine derivative, which is introduced in the plastic material. In particular, the at least one iodine derivative includes at least one oil-soluble organo-compound including for example iofendylate. Iofendylate (named also Pantopaque) is a mixture of isomers of ethyl iodophenylundecanoate and a radio-opaque organic compound containing 30.5% iodine. It is used particularly for myelography. As it is already widely used in medicine, there are no restrictions for its human use together with the plastic material that incorporates it. It cannot be used directly but it must first be incorporated into a plastic material to obtain a solid powder master. Subsequently, this master is added to the plastic material used to produce the device and/or thread material of the present invention.

Such agents, as known, are visible to X-rays and thus make it possible to monitor the position of the device to be implanted in the human body or the spacer device, as well as the material that contains them according to the present invention.

The material according to the present invention, moreover, can comprise further medical additives, like for example soluble and/or reabsorbable ceramic material, in the form of powder or granules, comprising tricalcium phosphate or calcium sulphate or hydroxyapatite, etcetera, or colouring substances of the biocompatible type and adapted to be introduced in the human body, etcetera.

Such additives, if they are not soluble or reabsorbable, can stay permanently inside the human body, or be removed if the biocompatible and implantable material in which they are contained is removed.

One of the problems to be solved, for the material according to the present invention, is that the substances contained therein can be degraded or undergo modifications due to the temperatures and/or pressures that the material according to the present invention encounters, when the material is heated or when it is moulded, for example through injection moulding or three-dimensional printing or another moulding technique.

In particular, the material according to the present invention can be in the form of a thread, for example having a diameter that can vary between 1 and 10 millimetres or between 1 and 5 millimetres or between 5 and 10 millimetres, for example adapted for being used to feed a three-dimensional printer of the thread or inkjet type or in another moulding technique.

One of the methods for allowing the material according to the present invention to be reduced to a thread adapted for being extruded and/or injected and/or printed three-dimensionally is as follows.

The base material is provided, for example in the form of a pellet (e.g. press pellet) or of granules; the material is inserted in suitable machinery, for example an extruder, and a pharmaceutical or medical substance and a radio-opacifying agent and/or a further additive is added.

Then the whole thing is heated until a certain temperature is reached, for example the melting temperature thereof, or to a temperature suitable for melting or softening the material in question (together with the possible mixture of additives that are added), to such a point as to be able to be extruded in threads or mouldable. Sometimes such temperature is higher than the melting temperature, while some other times it is slightly lower than the melting temperature.

Such a melting temperature varies as the polymers comprised according to the present invention varies. Most of such polymers have melting values comprised between 60° and 300°. Such a melting temperature can, for example, be around 250° C. One important point to be considered is that, while plastic material in general melts at the melting temperature, it is possible, especially for some medical or pharmaceutical substances, preferably antibiotics, that at the melting temperature they undergo degradation. Therefore, at the temperature wherein the plastic melts, the antibiotics lose their properties. This is the reason why the plastic melting temperature has to be lower than the melting/degradation temperature of the included medical or pharmaceutical substance, preferably antibiotic.

The at least one radiopacifying agent can be included at the time of forming the thread or the granules.

In a version of the invention, there is then a step of extruding the heated or molten material through suitable machinery, for example the same extruder, in the form of one or more threads having the diameter indicated above.

Such at least one thread can be wound in a coil so that, being extruded, it cools down and becomes consolidated and thus is suitable to then be handled and stored.

Such at least one thread can be used to feed a nozzle of an injection mould or a 3D printer.

Such a thread can be moulded through a three-dimensional printer or through injection moulding or through forming presses or through a thermoplastic moulding technique, in order to obtain a device that can be implanted in the human body or a spacer device for treating a bone or a joint location.

Such a method can, additionally or as an alternative to those described above, provides for a step of crushing or granulation of the base material, possibly after cooling thereof.

In a version of the invention, such a step is carried out through a suitable granulating machine.

Thereafter, the base material, or the crushed or granulated material, is used in a thermoplastic press with which moulded products are obtained.

In this case, in a version, a thread is not obtained before moulding, whereas in a second version, it is the thread obtained as indicated earlier that is crushed or granulated, possibly after cooling thereof.

The crushed or granulated material is then moulded, for example through an injection moulding press.

As stated, the temperature at which the material is melted must be below the degradation or damaging temperature of the pharmaceutical or medical substance, or of the radio-opacifying agent or of the further additive, so that they maintain their original characteristics also, following melting of the material, in the thread or resulting material.

Therefore, in a version of the invention, the extrusion or the moulding (but not the 3D printing) is carried out at lower temperatures than that at which the aforementioned substances degrade, by suitably selecting plastic materials with particularly low melting or softening temperatures, for example below the melting or degradation temperature of the pharmaceutical or medical substance, in order to obtain the end product comprising at least one from a pharmaceutical or medical substance, according to the specific requirements.

For example, an antibiotic that has a melting temperature of 180° C., so as not to degrade during moulding, must not exceed such a temperature.

Through the aforementioned material, it is possible to obtain devices that can be implanted in the human body like for example catheters, screws or joints, couplings or meshes, for which it may be useful to have a medicated version, a non-medicated version, an X-ray visible version, a coloured version, etcetera.

Alternatively, through the aforementioned material, it is possible to obtain devices that can be implanted in the human body like for example medical “threads”, for which it may be useful to have a medicated version, for example comprising an antibiotic and a radio-opaque substance.

Thanks to the present invention, if the material in question requires a melting temperature to obtain it in the form of thread or a moulding temperature above the degradation temperature of the pharmaceutical or medical substance, it will equally be possible to obtain the device in question, without the risk of damaging them.

In a further version, such devices are made with a material already added to with at least one pharmaceutical or medical substance but, being porous, once formed they can be capable of absorbing another substance, the same or different with respect to the one already contained in them.

In this way, it has been seen how the material according to the present invention, being able to be moulded and possibly being able to be made in the form of a thread, allows devices able to be implanted in the human body or spacer devices to be obtained in a quick, simple and possibly customizable manner both in terms of the shape and the size, and also the pharmaceutical or medical substances or the further additives or agents contained therein, according to the surgical or anatomical requirements of the patient.

The device that can be implanted in the human body, or to a spacer device for treating a bone or a joint location, comprises a material biocompatible and implantable in the human body according to the present invention.

Such devices, in fact, comprise an additive such as a pharmaceutical or medical substance and a radio-opacifying agent and/or a further additive, as described earlier for the material according to the present invention.

Such devices are made by moulding, for example through a three-dimensional printer or through injection moulding or through forming presses or through a thermoplastic moulding technique.

Another embodiment that can be obtained with the material according to the present invention is a cranial prosthesis. In this way, the prosthesis could be made directly from the CAT data, formed according to the configuration and the dimensions necessary for the anatomical and implanting requirements, and then implanted in the bone location of interest.

Such a cranial prosthesis could contain a pharmaceutical or medical substance and/or a radio-opacifying agent and/or a further additive.

As far as the thermal degradation of the active ingredients is concerned, the present invention is successfully able to lower the melting point of surgically used plastics such as UHMWPE. This ability is linked to the possibility to mix UHMWPE with a low molecular weight PE. In this way, in fact, it is possible to melt such mixture, make it extrudable and reduce it to a thread with still active antibiotics inside.

According to an example of the present invention, the plastic mixture comprises or consists of UHMWPE and at least one of a low-density PE and a high-density PE. Specifically, such plastic mixture comprises or consists of:
UHMWPE and low-density PE,
UHMWPE and a high-density PE, or
UHMWPE low-density PE and high-density PE.

In particular, UHMWPE in the mixture guarantees the mechanical properties of the resulting material, and therefore its percentage has not to go below a certain value. however, considering that the present invention is not aimed to provide permanent prosthesis, but temporary and/or disposable devices, the mechanical performances can be less stringent than for permanent prosthesis.

In the plastic mixture, UHMWPE can be present according to the following percentages: at least 50%, at least 60%, at least 70%, at least 80%, at least 90%, or preferably 55%, 65%, 75%, 85%, 95%. The higher the percentage of UHMWPE, the higher mechanical properties are present in the resulting material. However, the higher the percentage of UHMWPE, the lower is the flowability of the resulting material. Therefore, the specific percentage depends on the use and/or type of the resulting device.

The remaining percentage is given by the at least one of a low-density PE and a high-density PE. Preferably, when both components are present, they are present in an equal amount. Alternatively, it is preferably to have more low-density PE than high-density PE. Otherwise, it is preferably to have more high-density PE than low-density PE.

In particular, UHMWPE has a relative low melting temperature (135-140° C.), but it has a very low flowability and a high viscosity. In fact, melted UHMWPE is very viscous, and it does not really flow for example in a mould. In fact, it is usually worked mechanically, for example with a lathe. Its feature is to have poor flowability.

The mixture of UHMWPE and at least one of a low-density PE and a high-density PE is important to increase the flowability of the plastic material of the present invention. Therefore, the viscosity of the mixture is lower than the viscosity of the UHMWPE alone. In this way, it is easier to extrude (or to create a thread of) the plastic material or the mixture, with respect to the extrusion of the UHMWPE alone.

The melting temperature of the mixture can be affected or not by the presence of at least another PE, in addition to the UHMWPE.

Another way to safeguard antibiotics from thermal degradation, for example when it is not possible to reduce the melting temperature of the plastic material as in the case of PMMA thread (which has a transformation temperature of 230° C.), is to provide hollow microspheres, for example hollow glass microspheres or hollow ceramic microspheres or hollow silica microspheres, inside which to house the antibiotic/s and/or the other pharmaceutical or medical substances mentioned above. In this way, the antibiotic/s and/or the other pharmaceutical or medical substances mentioned above is/are hidden inside the microsphere, which protects it/them from thermal degradation during moulding.

The hollow microspheres can have an outer shell, for example in soluble material, enclosing an inner cavity, inside which the at least one antibiotic is inserted.

Otherwise, or in addition to the above, the hollow microspheres can be porous, and the shell or the entire volume of the microsphere presents small voids or pores, possibly interconnected.

At least one opening, therefore, has to be present in the microspheres, in order for them to be filled with the at least one antibiotic and/or other pharmaceutical or medical substance. This opening can be maintained open or can be closed before use.

Such microspheres have a reduced specific weight (when compared to wholly solid microspheres) and an increased heat resistance. In particular, the fact to be inside the microspheres allows to the antibiotic and/or the other pharmaceutical or medical substances mentioned above to thermo-resist (against degradations or denaturation) to temperatures used for extruding and/or melting the plastic material.

The microspheres can have a size of about 30-120 micron.

Furthermore, as an alternative or in addition to antibiotics, such hollow microspheres can be filled with pharmacological active ingredients in general. The active substances are kept inside the spheres, thus protecting them from degradation.

For example, the thermal resistance of the active ingredient is increased by about 30° C.

Another advantage of the present invention, when the material comprises UHMWPE, is that it does not adhere to the bone cement used to cement the device. In this way, even if it can cause some mechanical disadvantages, it results very improved when the device is temporary and/or disposal, because it is possible to remove it in an easy way (for example for catheters or spacer devices) and it allow, for example, screwing/connecting properties (for example when the device is a screw, joint, etc. to be used in the human body). Furthermore, the resulting material is resistant to breakage and it is flexible.

Another advantage can be conferred by the formation with such material of a mesh, for example for use at cranial level, in order to “trap” the bone cement inside the openings of the mesh and at the same time providing such mesh made of a flexible and resistant to breakage material, able to trap possible fragments derived from the bone cement.

The invention thus conceived can undergo numerous modifications and variants all covered by the inventive concept.

The characteristics presented for one version or embodiment can be combined with the characteristics of another version or embodiment, without departing from the scope of protection of the present invention.

Moreover, all of the details can be replaced by other technically equivalent elements. In practice, the materials used, as well as the contingent shapes and sizes, can be whatever according to requirements without for this reason departing from the scope of protection of the following claims.

1. A device implantable in the human body, including a temporary and/or disposable prosthetic device or spacer device for treating a bone or a joint location or a catheter, screw or joint, coupling or mesh, comprising a biocompatible material that can be implanted in the human body, wherein said biocompatible material comprises a plastic material and a pharmaceutical or medical substance including at least one antibiotic, wherein said biocompatible material comprises a radio-opacifying agent including at least one iodine derivative.

2. The device according to claim 1, wherein said iodine derivatives comprises at least one oil-soluble organo-compound including for example iofendylate and/or a mixture of isomers of ethyl iodophenylundecanoate and a radio-opaque organic compound containing about 30.5% iodine.

3. The device according to claim 1, wherein said radio-opacifying agent further comprises metallic powders or heavy metals powers, of tungsten, tantalum, silver metallic powders or a metal salt of barium sulphate, zirconium oxide, bismuth oxide.

4. The device according to claim 1, comprising hollow microspheres housing said at least one antibiotic inside an inner cavity thereof or inside pores thereof.

5. A device implantable in the human body, including a temporary and/or disposable prosthetic device or spacer device for treating a bone or a joint location or a catheter, screw or joint, coupling or mesh, comprising a biocompatible material that can be implanted in the human body, wherein said biocompatible material comprises a plastic material, a pharmaceutical or medical substance including at least one antibiotic and a radio-opacifying agent, and further comprising hollow microspheres housing said at least one antibiotic inside an inner cavity thereof or inside at least one of a plurality of pores thereof.

6. The device according to claim 5, wherein said radio-opacifying agent comprises at least one iodine derivative, including at least one oil-soluble organo-compound including for example iofendylate and/or a mixture of isomers of ethyl iodophenylundecanoate and a radio-opaque organic compound containing about 30.5% iodine and/or wherein said radio-opacifying agent comprises metallic powders or heavy metals powers, of tungsten, tantalum, silver metallic powders or a metal salt of barium sulphate, zirconium oxide, bismuth oxide.

7. The device according to claim 4, wherein said hollow microspheres comprise hollow glass microspheres or hollow ceramic microspheres or hollow silica microspheres, or microspheres including a shell and said inner cavity or porous microspheres comprising said pores, wherein said hollow microspheres have a size of about 30-120 microns.

8. The device according to claim 4, wherein the pores are interconnected.

9. The device according to claim 1, wherein said plastic material comprises or consists of an acrylic resin including a copolymer composed of MMA, styrene and ethyl acrylate or polymethylmethacrylate or mixtures comprising acrylic polymers and/or copolymers, or comprises or consists of polyethylene (PE) or low-density polyethylene or high-density polyethylene or ultra high molecular weight polyethylene (UHMWPE) or polypropylene or polyamide or polyetheretherketone (PEEK) or a mixture of UHMWPE and at least one of a low-density PE and a high-density PE, at least 50%, 60%, 70%, 80% or 90% of UHMWPE in such mixture, or a mixture of the above or wherein the plastic material is soluble and comprises polylactic or polyglycolic acid polymers or other suitable soluble polymers, or wherein said plastic material is selected to have a melting temperature which is lower than a degradation temperature of said at least one antibiotic.

10. The device according to claim 1, wherein said plastic material comprises a further additive including a soluble and/or reabsorbable ceramic material, in the form of powder or granules, comprising tricalcium phosphate or calcium sulphate or hydroxyapatite or a mixture thereof, and/or coloring substances of the biocompatible type and adapted to be introduced in the human body.

11. The device according to claim 1, wherein said at least one antibiotic comprises or consists of at least one of gentamicin sulphate, Amikacin, Azlocillin, Aztreonam, Clarithromycin, Chloramphenicol, Ciprofloxacin, Clindamycin, Coumermycin, Fosfomycin, Josamycin, Kanamycin, Mezlocillin, Mupirocin, Nalidixic acid, Netilmicin, Norfloxacin, Novobiocin, Ofloxacin, Oxacillin, Sulbactam, Tobramycin, Trimethoprim, Trimethoprim together with Sulphamethoxazole, Vancomycin, another suitable antibiotic which is thermoresistant and that is active against microbial infections, etc., and/or wherein said pharmaceutical or medical substance further comprises or consists of an antiseptic agent of organic or inorganic nature, a bacteriostatic agent comprising silver in its forms, such as metallic powder or salts comprising citrate, proteinate, colloidal, electrolytic, or other forms, or copper or gold in their forms or alloys, or as salts, boric acid.

12. A method for the obtainment of a device implantable in the human body, including a temporary and/or disposable prosthetic device or spacer device for treating a bone or a joint location or a catheter, screw or joint, coupling or mesh, comprising a biocompatible material that can be implanted in the human body, according to claim 1, comprising the following steps:
providing said biocompatible material which comprises a plastic material,
providing a pharmaceutical or medical substance including at least one antibiotic,
mixing said plastic material and said at least one antibiotic obtaining a mixed base material,
heating said mixed base material to a predetermined temperature obtaining a melted material,
extruding or forming said melted material obtaining at least one thread of material, or
cooling said melted material and then crushing or granulating it in order to obtain a crushed or granulated material, or
cooling said at least one thread of material and then crushing or granulating it in order to obtain a crushed or granulated material,
molding said at least one thread of material or said crushed or granulated material by means of a tridimensional printer or by means of injection molding or by means of molding press or using a thermoplastic molding technique, in order to obtain said device,
wherein said method comprises a step of providing a radio-opacifying agent including at least one iodine derivative,
and/or wherein said step of providing a pharmaceutical or medical substance including at least one antibiotic comprises providing hollow microspheres housing said at least one antibiotic inside an inner cavity thereof or inside pores thereof.

13. The method according to claim 12, wherein said predetermined temperature is below the degradation temperature of said at least one antibiotic.

14. The method according to claim 12, wherein said mixing step and said heating step of said material occur simultaneously in an extruder.

15. The method according to claim 12, wherein said molding step comprises feeding said at least one thread of material to a tridimensional printer in order to obtain said device.

16. The method according to claim 12, comprising a step of providing and mixing with said plastic material a further additive including a soluble and/or reabsorbable ceramic material, in the form of powder or granules, comprising tricalcium phosphate or calcium sulphate or hydroxyapatite or a mixture thereof, and/or coloring substances of the biocompatible type and adapted to be introduced in the human body, and/or comprising a step of providing said at least one antibiotic including at least one of gentamicin sulphate, Amikacin, Azlocillin, Aztreonam, Clarithromycin, Chloramphenicol, Ciprofloxacin, Clindamycin, Coumermycin, Fosfomycin, Josamycin, Kanamycin, Mezlocillin, Mupirocin, Nalidixic acid, Netilmicin, Norfloxacin, Novobiocin, Ofloxacin, Oxacillin, Sulbactam, Tobramycin, Trimethoprim, Trimethoprim together with Sulphamethoxazole, Vancomycin, another suitable antibiotic which is thermoresistant and that is active against microbial infections, etc.,
and/or said pharmaceutical or medical substance further comprising or consisting of an antiseptic agent of organic or inorganic nature, a bacteriostatic agent such as silver in its forms, such as metallic powder or salts comprising citrate, proteinate, colloidal, electrolytic, or other forms, or copper or gold in their forms or alloys, or as salts, boric acid.

17. A thread for the tridimensional moulding of a device according to claim 1, comprising a plastic material and a pharmaceutical or medical substance including at least one antibiotic, wherein said thread has a diameter comprised between 1 and 10 mm, wherein said thread comprises a radio-opacifying agent including at least one iodine derivative or hollow microspheres housing said at least one antibiotic inside an inner cavity thereof or inside pores thereof.

18. The thread according to claim 17, wherein said iodine derivatives comprises at least one oil-soluble organo-compound including iofendylate and/or a mixture of isomers of ethyl iodophenylundecanoate and a radio-opaque organic compound containing about 30.5% iodine.

19. The thread according to claim 17, wherein said at least one iodine derivative comprises at least one oil-soluble organo-compound including iofendylate and a mixture of isomers of ethyl iodophenylundecanoate and a radio-opaque organic compound containing about 30.5% iodine and wherein said radio-opacifying agent comprises metallic powders or heavy metals powers, of tungsten, tantalum, silver metallic powders or a metal salt of barium sulphate, zirconium oxide, bismuth oxide.

20. The thread according to claim 17, wherein said hollow microspheres are hollow glass microspheres or hollow ceramic microspheres or hollow silica microspheres, or microspheres including a shell and said inner cavity or porous microspheres comprising said pores, wherein said hollow microspheres have a size of about 30-120 microns.

21. The thread according to claim 17, wherein said plastic material comprises or consists of an acrylic resin including a copolymer composed of MMA, styrene and ethyl acrylate or polymethylmethacrylate or mixtures comprising acrylic polymers and/or copolymers, or comprises or consists of polyethylene (PE) or low-density polyethylene or high-density polyethylene or ultra high molecular weight polyethylene (UHMWPE) or polypropylene or polyamide or polyetheretherketone (PEEK) or a mixture of UHMWPE and at least one of a low-density PE and a high-density PE, at least 50%, 60%, 70%, 80% or 90% of UHMWPE in such mixture, or a mixture of the above or wherein the plastic material is soluble and comprises polylactic or polyglycolic acid polymers or other suitable soluble polymers, or wherein said plastic material is selected to have a melting temperature which is lower than a degradation temperature of said at least one antibiotic.

22. The thread according to claim 17, wherein said plastic material comprises a further additive including a soluble and/or reabsorbable ceramic material, in the form of powder or granules, comprising tricalcium phosphate or calcium sulphate or hydroxyapatite or a mixture thereof, and/or coloring substances of the biocompatible type and adapted to be introduced in the human body.

23. A method for the obtainment of a device implantable in the human body, including a temporary and/or disposable prosthetic device or spacer device for treating a bone or a joint location or a catheter, screw or joint, coupling or mesh, comprising a biocompatible material that can be implanted in the human body, according to claim 5, comprising the following steps:
providing said biocompatible material which comprises a plastic material,
providing a pharmaceutical or medical substance including at least one antibiotic,
mixing said plastic material and said at least one antibiotic obtaining a mixed base material,
heating said mixed base material to a predetermined temperature obtaining a melted material,
extruding or forming said melted material obtaining at least one thread of material, or
cooling said melted material and then crushing or granulating it in order to obtain a crushed or granulated material, or
cooling said at least one thread of material and then crushing or granulating it in order to obtain a crushed or granulated material,
molding said at least one thread of material or said crushed or granulated material by means of a tridimensional printer or by means of injection molding or by means of molding press or using a thermoplastic molding technique, in order to obtain said device,
wherein said method comprises a step of providing a radio-opacifying agent including at least one iodine derivative,
and/or wherein said step of providing a pharmaceutical or medical substance including at least one antibiotic comprises providing hollow microspheres housing said at least one antibiotic inside an inner cavity thereof or inside pores thereof.
